(19) United States
(12) Patent Application Publication
(10) Pub. No.: US 2023/0218229 A1
(43) Pub. Date: Jul. 13, 2023

(54) MONITORING PATCH

(71) Applicant: ABIOMED, Inc., Danvers, MA (US)

(72) Inventors: Jerald Curran, Danvers, MA (US); Samantha Polak, Danvers, MA (US); Matt Gaylord, Danvers, MA (US); Jin Kwang Kim, Danvers, MA (US); Erik Ian Kroeker, Danvers, MA (US)

(73) Assignee: ABIOMED, Inc., Danvers, MA (US)

(21) Appl. No.: 18/093,850

(22) Filed: Jan. 6, 2023

Related U.S. Application Data
(60) Provisional application No. 63/296,992, filed on Jan. 6, 2022.
(60) Provisional application No. 63/346,091, filed on May 26, 2022.

(51) Int. Cl. A61B 5/00; A61B 5/145; A61B 5/28; A61B 5/01

(52) U.S. Cl. CPC A61B 5/6833; A61B 5/14517; A61B 5/28; A61B 5/01; A61B 2562/164

(57) ABSTRACT

A monitoring patch including a substrate having an adhesive surface and a plurality of sensors disposed in and/or on the substrate. In some embodiments, the plurality of sensors may include one or more sensors arranged to measure oxygen saturation, a lactate sensor, and one or more impedance cardiography electrodes. In some embodiments, the plurality of sensors may include an accelerometer and a strain gauge, and may be free of at least one sensor of a sensor arranged to measure oxygen saturation, a lactate sensor, or an impedance cardiography electrode.

### CROSS-REFERENCE TO RELATED APPLICATIONS

The application claims priority to U.S. Provisional Patent App. Nos. 63/296,992, filed Jan. 6, 2022, and 63/346,091, filed May 26, 2022, the contents of each of which are each incorporated by reference herein in their entirety.

### TECHNICAL FIELD

The present disclosure relates to patient monitoring, and more specifically to patient monitoring devices configured to monitor physiological conditions of a subject, (e.g., pulse rate, oxygenation levels, etc.).

### BACKGROUND

Patients who are undergoing a medical procedure, or who have recently had such a procedure, are often monitored to ensure various physiological conditions are within acceptable parameters. Current techniques for short-term (e.g., during the procedure and a short period of time afterwards) or long-term (e.g., several weeks or months) monitoring often involve invasive and/or costly devices, and the long-term monitoring may involve different devices than those used for short-term monitoring.

Mechanical circulatory support (MCS) devices, such as ventricular assist device (VADs) and catheter-based ventricular assist devices (such as intravascular blood pumps) may be used to mechanically unload the heart, such as the left ventricle (e.g., reducing the left ventricular volume, which results in pressure reduction) and/or decompression of the left ventricle (e.g., volume reduction of the left ventricle, which may be driven by a hole in the wall between the left atrium and right atrium resulting in a lower preload of the left ventricle)).

### BRIEF SUMMARY

A monitoring patch may include a substrate having an adhesive surface and a plurality of sensors disposed in and/or on the substrate.

According to a first aspect of the present disclosure, the plurality of sensors may include: one or more sensors arranged to measure oxygen saturation; a lactate sensor; and one or more impedance cardiography electrodes.

In some embodiments, the monitoring patch may also include a sweat biosensor array. In some embodiments, the sweat biosensor array may be configured to detect Na⁺, K⁺, or a combination thereof In some embodiments, the sweat biosensor array may be configured to detect at least pH, glucose, lactate, an alkali metal ion, an alkaline earth metal ion, or a combination thereof. In some embodiments, the sweat biosensor array may be configured to measure sodium, pH, potassium, glucose, lactate, or a combination thereof.

In some embodiments, the lactate sensor may include a sub cutaneous lactate sensor. In some embodiments, the substrate may comprise a first side and a second side opposite the first side, where the lactate sensor is positioned on the first side of the substrate. In some embodiments, the monitoring patch may also include a power source or a connection for a power source, which may be disposed on the second side. In some embodiments, the one or more impedance cardiology electrodes may be disposed on the second side.

In some embodiments, the monitoring patch may also include a temperature sensor. In some embodiments, the monitoring patch may also include an electrocardiogram (ECG) surface electrode. In some embodiments, the monitoring patch may also include a strain gauge. In some embodiments, the monitoring patch may also include an accelerometer.

In some embodiments, the one or more sensors arranged to measure oxygen saturation may include NIR emitter/detectors, IR emitter/detectors, or both.

In some embodiments, the monitoring patch may also include circuitry on a second surface of the substrate, the second surface being opposite the first surface, the circuitry being operably connected to the plurality of different sensors on the first surface and comprising a connector for connecting to a power source. In some embodiments, the power source may be operably coupled to the substrate. In some embodiments, the power source may be remotely located from the patch.

In some embodiments, the monitoring patch may include a detachable wireless transmitter or transceiver configured to operably connect to the circuitry. In some embodiments, the detachable wireless transmitter or transceiver may be a detachable Bluetooth transmitter or transceiver.

In some embodiments, the monitoring patch may be configured to connect to a mobile phone, a tablet, or a desktop computer. In some embodiments, the monitoring patch may be configured to operably communicate with a remote server.

In some embodiments, the substrate may be configured to be bilaterally symmetrical, having a left portion and a right portion. In some embodiments, each impedance cardiography electrode may have a portion on a first surface of the substrate and a portion on a second surface of the substrate, the second surface being opposite the first surface.

In some embodiments, the substrate may be configured to be bilaterally symmetrical, having a left portion and a right portion, where a first impedance cardiography electrode is within the left portion and a second impedance electrode is within the right portion.

In some embodiments, the substrate may be comprised of two or more layers.

According to a second aspect of the present disclosure, the plurality of sensors may

include: an accelerometer and a strain gauge, where the monitoring patch is free of at least one of a sensor arranged to measure oxygen saturation, a lactate sensor, and/or an impedance cardiography electrode.

In some embodiments, the monitoring patch may also include circuitry on a second surface of the substrate, the second surface being opposite the first surface, the circuitry being operably connected to the plurality of different sensors on the first surface and comprising a connector for connecting to a power source. In some embodiments, the power source may be operably coupled to the substrate. In some embodiments, the power source may be remotely located from the patch.

In some embodiments, the monitoring patch may include a detachable wireless transmitter or transceiver configured to operably connect to the circuitry. In some embodiments, the detachable wireless transmitter or transceiver may be a detachable Bluetooth transmitter or transceiver.

In some embodiments, the monitoring patch may be configured to connect to a mobile phone, a tablet, or a desktop computer. In some embodiments, the monitoring patch may be configured to operably communicate with a remote server.

In some embodiments, the monitoring patch may also include a sweat biosensor array. In some embodiments, the sweat biosensor array may be configured to detect Na⁺, K⁺, or a combination thereof In some embodiments, the sweat biosensor array may be configured to detect at least pH, glucose, lactate, an alkali metal ion, an alkaline earth metal ion, or a combination thereof. In some embodiments, the sweat biosensor array may be configured to measure sodium, pH, potassium, glucose, lactate, or a combination thereof.

In some embodiments, the monitoring patch may also include a temperature sensor. In some embodiments, the monitoring patch may also include an electrocardiogram (ECG) surface electrode.

In some embodiments, the monitoring patch may include a lactate sensor. In some embodiments, the lactate sensor may include a sub cutaneous lactate sensor. In some embodiments, the substrate may comprise a first side and a second side opposite the first side, where the lactate sensor is positioned on the first side of the substrate.

In some embodiments, the monitoring patch may also include a power source or a connection for a power source, which may be disposed on the second surface.

In some embodiments, the monitoring patch may include one or more impedance cardiology electrodes disposed in and/or on the substrate. In some embodiments, the one or more impedance cardiology electrodes may be disposed on the second surface. In some embodiments, each impedance cardiography electrode may have a portion on the first surface and a portion on the second surface of the substrate.

In some embodiments, the monitoring patch may include one or more sensors arranged to measure oxygen saturation. In some embodiments, the one or more sensors arranged to measure oxygen saturation may include NIR emitter/detectors, IR emitter/detectors, or both.

In some embodiments, the substrate may be configured to be bilaterally symmetrical, having a left portion and a right portion. In some embodiments, each impedance cardiography electrode may have a portion on a first surface of the substrate and a portion on a second surface of the substrate, the second surface being opposite the first surface.

In some embodiments, the substrate may be configured to be bilaterally symmetrical, having a left portion and a right portion, where a first impedance cardiography electrode is within the left portion and a second impedance electrode is within the right portion.

In some embodiments, the substrate may be comprised of two or more layers. In some embodiments, a system for combination use may be provided. The system may include an embodiment of a monitoring patch as disclosed herein, operably coupled to a patient; and a mechanical circulatory support device operably coupled to the patient. In some embodiments, the mechanical circulatory support device may be a percutaneous blood pump.

According to a third aspect of the present disclosure, a monitoring patch kit may have a plurality of patches, including a first patch that is a monitoring patch according to any one of embodiments described herein, and a second patch.

In some embodiments, the second patch may include a substrate having an adhesive surface and a plurality of different sensors disposed in and/or on the substrate of the second patch, where at least one of the plurality of sensors disposed in and/or on the substrate of the first patch is different from any of the plurality of different sensors disposed in and/or on the substrate of the second patch.

In some embodiments, the monitoring patch kit may include a third and fourth patch, where each of the third and fourth patches may be a monitoring patch according to any one of the embodiments described herein.

In some embodiments, the monitoring patch kit may include a third and fourth patch, wherein each of the third and fourth patches has a substrate having an adhesive surface and a plurality of different sensors disposed in and/or on the substrate of each of the third and fourth patch, where at least one of the plurality of sensors disposed in and/or on the substrate of the first patch is different from any of the plurality of different sensors disposed in and/or on the substrate of the third patch and from any of the plurality of different sensors disposed in and/or on the substrate of the fourth patch.

In some embodiments, the first patch may be configured to be placed on a first portion of a subject's body and the second patch may be configured to be placed on a different portion of the subject's body. In some embodiments, the first portion of the subject's body may be a torso, thigh, or a combination thereof. In some embodiments, the different portion may be a forearm, wrist, neck, calf, ankle, or combination thereof.

In some embodiments, the first or second patch is configured to be placed on a subject's torso, upper arm, thigh, neck, or combination thereof, and wherein the other of the first or second patch is configured to be placed on a subject's forearm, wrist, calf, ankle, or combination thereof.

In some embodiments, the monitoring patch kit includes at least two patches configured to be placed on a torso, and at least one patch configured to be placed on a thigh. In some embodiments, the monitoring patch kit includes at least one patch configured to be placed on a left calf or ankle, and at least one patch configured to be placed on a right calf or ankle. In some embodiments, the monitoring patch kit includes at least one patch configured to be placed on a neck.

In some embodiments, the monitoring patch kit may include one or more additional patches including a first additional patch having a first set of different sensors disposed in and/or on a first substrate, and a second additional patch having a second set of different sensors disposed in and/or on a second substrate, where the first set of different sensors is different than the second set of different sensors, the first substrate is different from the second substrate, and/or a shape of the first substrate is different than a shape of the second substrate.

In some embodiments, the monitoring patch kit may include a controller, the controller configured to receive data from sensors on each of the plurality of patches. In some embodiments the controller is operably connected to a wireless transceiver. In some embodiments, the monitoring patch kit may include a plurality of leads, each lead configured to operably connect at least one sensor on one of the plurality of patches to the controller.

In some embodiments, the first patch and the second patch are configured to communicate with a controller and/or with each other.

According to a fourth aspect of the present disclosure, a method may include receiving data from one or more sensors on one or more monitoring patches according to any one of the embodiments described herein, the one or more monitoring patches being positioned on a subject.

In some embodiments, the method may include storing the received data on one or more non-transitory computer readable media, displaying the received data on a display, or a combination thereof. In some embodiments, the method may include transmitting the data to a remote server, a mobile device, a patient console, or a combination thereof. In some embodiments, the method may include determining whether the data received from the sensor is outside a first predetermined range. In some embodiments, the method may include displaying a value represented by the received data. In some embodiments, the value may be an oxygenation level, a lactate level, a glucose level, a potassium level, a sodium level, a pulse rate, a pH, and/or a temperature.

In some embodiments, differences between measured values may be determined. In some embodiments, the data may include a first value from a sensor on a first patch and a second value from a sensor on the second patch, and the method may include determining a first difference between the first value and the second value at a first point in time. In some embodiments, the data may include a first value from a first sensor on a first patch and a second value from a second sensor of on the first patch, and the method may include determining a first difference between the first value and the second value at a first point in time. In some embodiments, the method may include determining whether the first difference is outside a second predetermined range.

In some embodiments, the method further comprises determining a second difference between the first value and the second value at a second point in time. In some embodiments, the first value is a first pulse value and a second value is a second pulse value.

In some embodiments, the method may include alerting a subject and/or medical professional when the received data is outside the first predetermined range and/or the second predetermined range. In some embodiments, alerting the subject and/or medical professional may include transmitting an alert to a remote server, a desktop computer, a laptop, a mobile device, a patient console, or a combination thereof. In some embodiments, alerting the subject and/or medical professional may include generating an audible indicium, a visual indicium, a message, or a combination thereof. In some embodiments, the visual indicium may include a visual change to a display screen. In some embodiments, the method may include automatically determining and proposing a treatment plan based on the received data, the first difference, a change from the first difference to the second difference, or a combination thereof.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A and 1B are schematics of an embodiment of a monitoring patch, showing details of a first side (1A) and second side (1B) of the patch.

FIG. 2 is a representation of an embodiment of a monitoring patch communicating with various components.

FIGS. 3A-3D are 2D illustrations of different configurations of embodiments of substrates.

FIG. 4 is an illustration of an embodiment of a substrate with multiple layers.

FIGS. 5A-5H are 2D illustrations of different embodiments of sensor configurations on a monitoring patch.

FIG. 6 is an illustration of an embodiment of a monitoring patch kit.

FIG. 7A illustrates an embodiment in which one or more monitoring patches are affixed to a patient.

FIG. 7B illustrates another embodiment in which one or more patches are affixed to a patient receiving mechanical circulatory support.

FIG. 8 illustrates a method according to one embodiment.

FIG. 9 illustrates an example of a percutaneous blood pump according to one embodiment.

### DETAILED DESCRIPTION

Patients who are undergoing a medical procedure, who have recently had such a procedure, or who are scheduled to undergo a medical procedure may be monitored to ensure various physiological conditions are within acceptable parameters. Patients also may be monitored to determine if a medical treatment is needed and/or of a medical treatment may be changed or discontinued (e.g., if the patient has recovered to a desired degree).

Current techniques for short-term (e.g., during a medical procedure or treatment and a short period of time afterwards) or long-term (e.g., several weeks or months) monitoring often involve invasive and/or costly devices, and/or may require multiple devices to be able to monitor all the desired parameters.

In view of the above, the inventors have recognized the benefit of a non-invasive monitoring system capable of monitoring multiple parameters (e.g., of the patient). In some embodiments, the monitoring system includes a monitoring patch having one or more sensors arranged to monitor one or more parameters of the patient, such as oxygen saturation, lactate, glucose, temperature, heart rate, pH, and/or potassium. For example, in some embodiments, one or more monitoring patches may be placed on various portions of a patient's body to monitor one or more parameters. In some embodiments, the system may include a kit with multiple monitoring patches. In some embodiments, the monitoring patches in the kit may be the same (e.g., each have the same sensors) or the monitoring patches may be different (e.g., have different sensors on some of the patches). In such embodiments, a clinician may decide which monitoring patches to adhere to the patient (and at what location) to monitor the desired parameters.

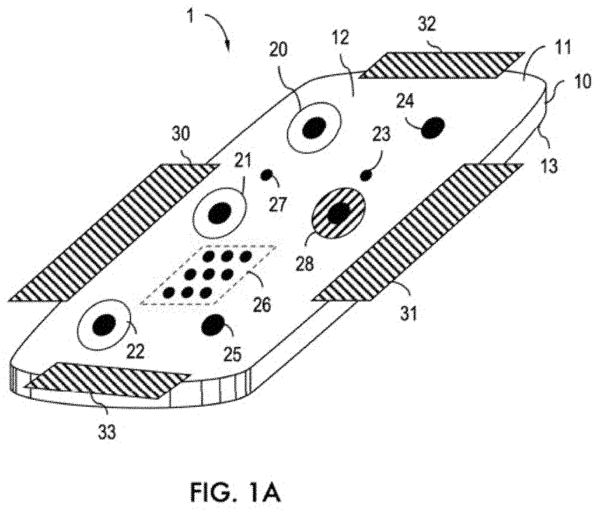

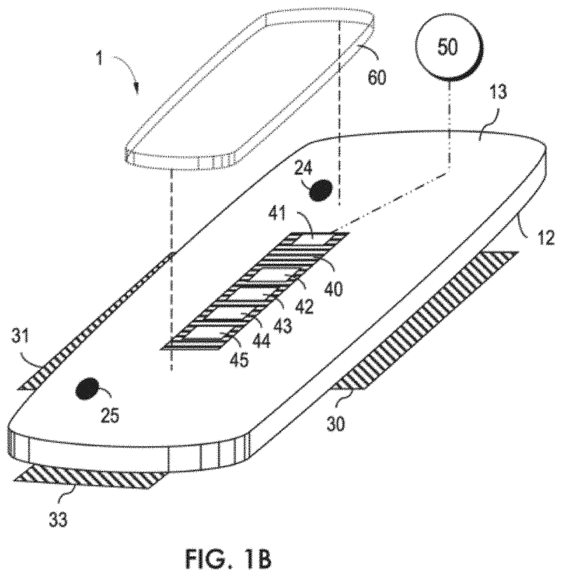

Referring now to the figures, FIGS. 1A and 1B, show a monitoring patch 1 according to embodiments of the present disclosure. As shown in these views, the monitoring patch may include a substrate 10 having an adhesive surface 11 and a plurality of sensors (20-28, 30, 31) disposed in and/or on the substrate 10.

In some embodiments of the present disclosure, the plurality of sensors may include: one or more sensors arranged to measure oxygen saturation 20, 21, 22; a lactate sensor 23; and one or more impedance cardiography electrodes 24, 25 arranged to monitor mechanical function of the heart.

In some embodiments, the substrate may comprise a first side 12 (or first surface) and a second side 13 (or second surface) opposite the first side (or first surface). In some embodiments, the first side 12 is the side of the substrate 10 that faces and is attachable to a subject's (e.g., patient's) skin. In some embodiments, each sensor arranged to measure oxygen saturation 20, 21, 22 is positioned on the first side of the substrate. In some embodiments, the lactate sensor 23 is also positioned on the first side of the substrate. In some embodiments, at least a portion of the one or more impedance cardiography electrodes 24, 25 are positioned on the first side of the substrate. As will be appreciated, the sensors may be disposed in other suitable manners in or on the substrate in other embodiments.

In some embodiments, the one or more impedance cardiography electrodes 24, 25 may comprise or consist of a plurality of impedance cardiography electrodes 24, 25. In some embodiments, the one or more impedance cardiology electrodes 24, 25 may be at least partially disposed on the second side. In some embodiments, each impedance cardiography electrode may have a portion on a first side 12 (or surface) of the substrate and another portion on a second side 13 (or surface) of the substrate.

In some embodiments, the one or more sensors arranged to measure oxygen saturation 20, 21, 22 may comprise or consist of NIR emitter/detectors, IR emitter/detectors, or both. In some embodiments, the monitoring patch may include a plurality of sensors for measuring oxygen saturation, where each sensor is an NIR emitter/detector. In some embodiments, the monitoring patch may include a plurality of sensors for measuring oxygen saturation, where each sensor is an IR emitter/detector. In some embodiments, the monitoring patch may include a plurality of sensors for measuring oxygen saturation (e.g., StO₂/SmO₂/TSI), where at least one of the plurality of sensors is an NIR emitter/detector, and at least one of the plurality of sensors is an IR emitter/detector. In some embodiments, the one or more sensors arranged to measure oxygen saturation may comprise or consist of at least three sensors. In some embodiments, the one or more sensors may measure light intensity difference after absorption and scattering. In some embodiments, the detectors may indicate changes in HbO₂. In some embodiments, the light may travel in a banana shape. In some embodiments, the penetration may include between ½ and 1/3 of source-detector separation, although other suitable penetration depths may be used. In some embodiments the IR emitter/detector may include gel and adhesive around the sensor.

In some embodiments, the lactate sensor 23 may comprise or consist of a subcutaneous lactate sensor.

In some embodiments, one or more additional sensors may be incorporated in the monitoring patch, such as in or on the substrate (e.g., on or in the first side of the substrate). For example, in some embodiments, the monitoring patch may also include a sweat biosensor array 26. In some embodiments, the sweat biosensor array 26 may be positioned on the first side of the substrate. In some embodiments, the sweat biosensor array may be configured to detect sodium (Na⁺), potassium (K⁺), or a combination thereof. In some embodiments, the sweat biosensor array may be configured to detect at least pH, glucose, lactate, an alkali metal ion, an alkaline earth metal ion, or a combination thereof. In some embodiments, the sweat biosensor array may be configured to measure sodium, pH, potassium, glucose, lactate, or a combination thereof. As will be appreciated, the blood chemistry may be used to indicate one or more conditions of the patient. For example, in some embodiments, the lactate sensor may be configured to measure the level of blood lactate, which may indicate muscle fatigue (e.g., cardiac muscle fatigue) in some embodiments. As described herein, such patient information may be used to determine a level of patient recovery and/or a need for further or new treatment.

In some embodiments, the monitoring patch may also include a temperature sensor 27. In such embodiments, the temperature sensor 27 also may be positioned on the first side of the substrate.

In some embodiments, the monitoring patch may also include an electrocardiogram (ECG) surface electrode 28 arranged to monitor a patient's heart rate. In some embodiments, the ECG surface electrode 28 is positioned on the first side of the substrate. In some embodiments, the ECG surface electrode may include an electrode and a gel and adhesive around the electrode.

In some embodiments, the monitoring patch may also include a strain gauge 30, 31, 32, 33. In some embodiments, the monitoring patch may include only a single strain gauge. In other embodiments a pair of strain gauges may be present. In some embodiments, each strain may extend beyond an edge of the substrate. For example, in some embodiments, each strain gauge of the pair of strain gauges may extend beyond an edge of the substrate on opposite sides of the substrate (e.g., strain gauges 30, 31 or strain gauges 32, 33). In some embodiments, each strain gauge of the pair of strain gauges may extend beyond an edge of the substrate on adjacent sides of the substrate (e.g., strain gauges 30, 32 or strain gauges 30, 33). In some embodiments, each pair of strain gauges are configured to measure strain in parallel directions (that is, both gauges of a pair are configured to measure strain cross-wise or length-wise with respect to the substrate). In some embodiments, four strain gauges 30, 31, 32, 33 may be present.

As is known in the art, strain gauges can compress and expand, and as they do so, a measurable characteristic (such as resistance) changes, which may be indicative of expansion (or contraction). Accordingly, in some embodiments, the one or more strain gauges may be arranged to measure swelling and/or stretching of a portion of a patient's body, such as the skin, of the patient's hand, arms, fee, ankles, and/or legs. In some embodiments, the strain gauge(s) may monitor, and in some embodiments measure, swelling and/or expansion of the patient's body, such as due to retaining a fluid volume or bloating. In some embodiments, the strain gauge(s) on the monitoring patch may be able to indicate a medical condition (e.g., heart failure) or an issue with a prescribed medication.

In some embodiments, the monitoring patch may also include an accelerometer 43. In some embodiments, the accelerometer may include a six-axis accelerometer. In other embodiments, the accelerometer may include a three-axis accelerometer. In still other embodiments, the accelerometer may include a two-axis accelerometer. In some embodiments, the accelerometer may be arranged to monitor movement of the patient. For example, the accelerometer may indicate when a patient is up and walking and/or if a patient falls.

In some embodiments, the monitoring patch 1 may also include circuitry 40, which may be positioned on a second side 13 of the substrate, the second side 13 being opposite the first side 12, the circuitry 40 being operably connected to at least one of the plurality of different sensors 20-28, 30-33, 43. In some embodiments, the circuitry 40 is operably connected to all of the plurality of different sensors 20-28, 30-33, 43. In some embodiments, the circuitry 40 is operably connected to the plurality of different sensors 20-28, 30-33 on the first surface. The circuitry also may be connected to the plurality of different sensors on the second surface.

In some embodiments, the circuitry 40 comprises a connector 41 for connecting to a power source 50. In some embodiments, the power source may be a separate battery pack or other medically acceptable DC power source, although other suitable power sources may be used in other embodiments. In some embodiments, the power source may be operably coupled to the substrate. For example, a coin cell or battery pack may be mounted directly onto the substrate. In some embodiments, the power source may be remotely located from the patch.

In some embodiments, the circuitry 40 may comprise one or more processors 44 and one or more non-transitory computer-readable storage media 45 (e.g., flash memory, etc.).

In some embodiments, the monitoring patch 1 may include a detachable wireless transmitter or transceiver 60 configured to operably connect to the circuitry. In some embodiments, the detachable wireless transmitter or transceiver may be a detachable Bluetooth transmitter or transceiver.

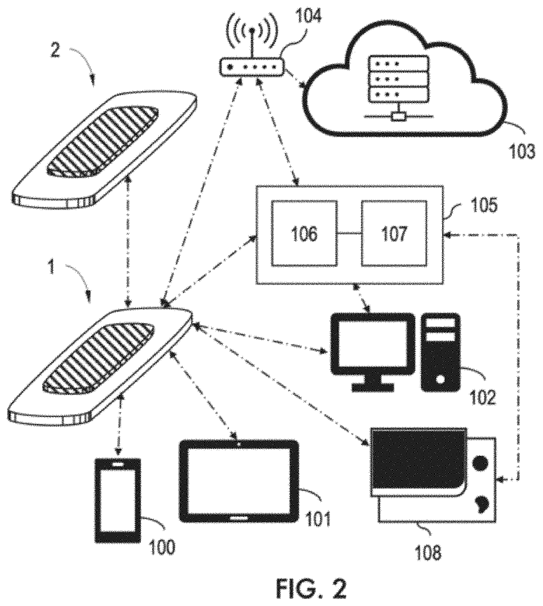

As seen in FIG. 2, in some embodiments, the monitoring patch 1 may be configured to connect to a mobile device, such as a mobile phone 100 and/or a tablet 101, a computer (e.g., desktop computer 102 and/or laptop), and/or to a patient console 108. In some embodiments, the monitoring patch 1 may be configured to operably communicate with a remote server 103, e.g., a cloud-based server, such as through one or more routers 104. As will be appreciated, the remote server also may be operably connected to one or more of the mobile devices, computers, and/or patient console. As will be appreciated, the monitoring patches also may be directly connected to the patient console in other embodiments. In some embodiments, the console (and/or the cloud) may include an integrated dashboard.

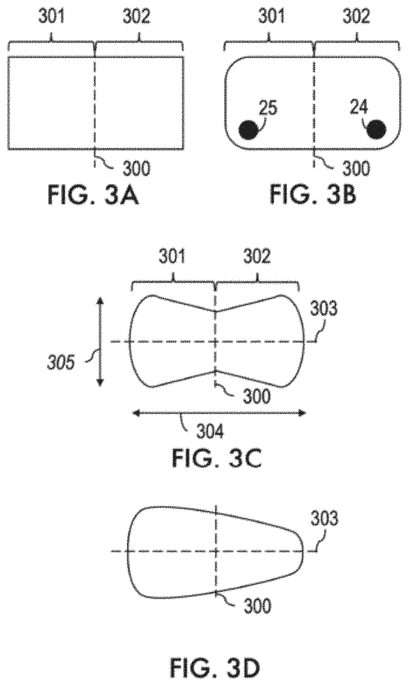

As seen in FIGS. 3A-3C, in some embodiments, the substrate may be configured to be bilaterally symmetrical around a central plane 300, having first and second portions, e.g., a left portion 301 and a right portion 302. As shown in FIG. 3B, in some embodiments, a first impedance cardiography electrode 25 may be located within the left portion 301 of the substrate and a second impedance electrode 24 may be located within the right portion 302 of the substrate. As seen in FIG. 3C, in some embodiments, the substrate may be configured to be bilaterally symmetrical around a central plane 300 normal to the surface that bisects the length 304 of the substrate, as well as around a central plane 303 normal to the surface that bisects the width 305 of the substrate.

In some embodiments, the substrate may be configured to be bilaterally asymmetrical in a first direction, but be bilaterally symmetrical in a perpendicular direction. For example, as seen in FIG. 3D, the substrate may be bilaterally asymmetrical around a central plane 300 normal to the surface that is oriented in a direction that bisects the length of the substrate, but well as around a central plane 303 normal to the surface that is oriented in a direction that is perpendicular to the length, and bisects the width of the substrate.

As will be appreciated, the substrate may have other suitable shapes and sizes in other embodiments. As will be further appreciated, in some embodiments, a first monitoring patch may have a first shape and size and a second monitoring patch may have a second shape and size. For example, in some embodiments, a kit may include multiple monitoring patches having different shapes and sizes. In such embodiments, the patches may be sized and shaped depending upon the portion of the body on which the patch is to be attached.

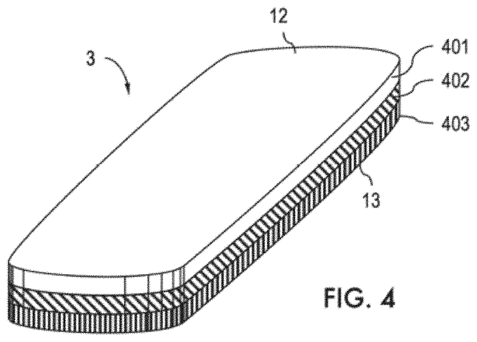

In some embodiments, the substrate may include a plurality of layers, such as two or more layers. As seen in FIG. 4, in some embodiments, substrate 3 may include a first layer 401, a second layer 402, and a third layer 403. In some embodiments, the substrate may be comprised of two layers (e.g., 401, 402). In some embodiments, the substrate may be comprised of three layers (e.g., 401, 402, 403). In some embodiments, additional layers may be included. For example, in some embodiments, each layer may include an extensible and/or elastomeric material. In some embodiments, at least one layer may include an extensible and/or elastomeric material. In some embodiments, the substrate is free of an extensible and/or elastomeric material. For purposes herein, the terms “elastomeric” and “extensible” refer to materials that extend in at least one direction when a force is applied to the material, and that return to approximately their original dimension(s) after the force is released. In some embodiments, at least one layer may include a nonwoven material. In some embodiments, at least one layer may include a foam material.

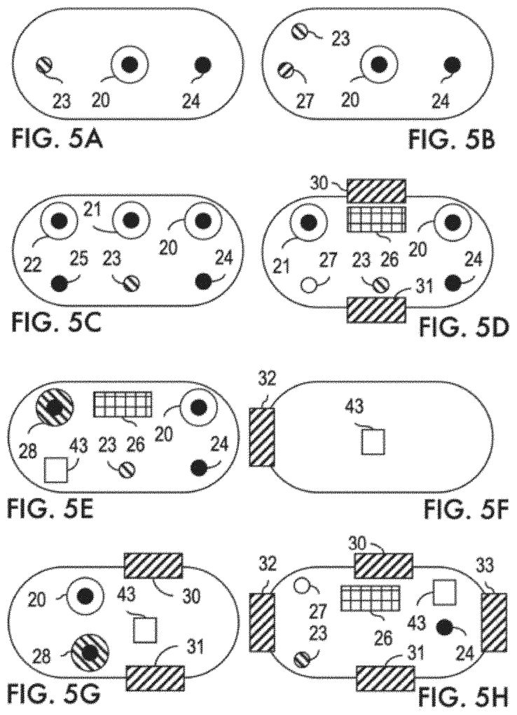

Any combination of sensors is envisioned, numerous non-limiting examples of which can be seen in FIGS. 5A-5H.

As described herein, in some embodiments, the monitoring patches may include one or more sensors arranged to measure oxygen saturation, a lactate sensor, and one or more impedance cardiography electrodes, with other sensors, such as an accelerometer and a strain gauge being optional. For example, in FIG. 5A, the monitoring patch may include only a sensor arranged to measure oxygen saturation 20, a lactate sensor 23, and an impedance cardiography electrode 24. In FIG. 5B, a temperature sensor 27 may be added to the three sensors from FIG. 5A. As shown in FIG. 5C, the monitoring patch may include three sensors arranged to measure oxygen saturation 20, 21, 22, a lactate sensor 23, and two impedance cardiography electrodes 24, 25. As shown in FIG. 5D, the monitoring patch may include two sensors arranged to measure oxygen saturation 20, 21, a lactate sensor 23, an impedance cardiography electrodes 24, a sweat biosensor array 26, a temperature sensor 27, and strain gauges 30, 31. As shown in FIG. 5E, the monitoring patch may include a sensor arranged to measure oxygen saturation 20, a lactate sensor 23, an impedance cardiography electrodes 24, a sweat biosensor array 26, an ECG surface electrode 28, and an accelerometer 43.

In other embodiments, the monitoring patches may include an accelerometer and a strain gauge, with other sensors being optional. In some embodiments, the monitoring patch may be free of a sensor arranged to measure oxygen saturation, a lactate sensor, and/or an impedance cardiography electrode. This can be seen in FIGS. 5F-5H, for example. In FIG. 5F, the patch is shown as having only an accelerometer 43 and a single strain gauge 32. In FIG. 5G, the patch is shown as having an accelerometer 43 and two strain gauges 30, 31, a sensor arranged to measure oxygen saturation 20, and an ECG surface electrode 28 (but no lactate sensor or impedance cardiography electrode). In FIG. 5H, the patch is shown as comprising only an accelerometer 43 and four strain gauges 30, 31, 32, 33, a lactate sensor 23, an impedance cardiography electrodes 24, a sweat biosensor array 26, and a temperature sensor 27 (but no sensor arranged to measure oxygen saturation). As will be appreciated in view of the present disclosure, the number and type of sensors on the monitoring patch may be selected based upon the type of monitoring desired and the specific location where the patch is to be placed on the patient.

In some embodiments, the patches shown in FIGS. 5A-5H may be otherwise identical to those described herein (e.g., with respect to the size, shape, number of layers). For example, as described herein, in some embodiments, the monitoring patch may also include circuitry on a second surface of the substrate, the second surface being opposite the first surface. The circuitry may be operably connected to some or all of the plurality of different sensors, as described herein. In some embodiments, the circuitry comprises a connector for connecting to a power source. In some embodiments, the power source may be operably coupled to the substrate. In some embodiments, the power source may be remotely located from the patch. As described herein, in some embodiments, the monitoring patch may include a detachable wireless transmitter or transceiver configured to operably connect to the circuitry. In some embodiments, the detachable wireless transmitter or transceiver may be a detachable Bluetooth transmitter or transceiver.

As also described herein, in some embodiments, the monitoring patch may be configured to connect to a mobile phone, a tablet, or a desktop computer. In some embodiments, the monitoring patch may be configured to operably communicate with a remote server.

As also described herein, in some embodiments, the monitoring patch may also include a sweat biosensor array. In some embodiments, the sweat biosensor array may be configured to detect Na⁺, K⁺, or a combination thereof. In some embodiments, the sweat biosensor array may be configured to detect at least pH, glucose, lactate, an alkali metal ion, an alkaline earth metal ion, or combinations thereof. In some embodiments, the sweat biosensor array may be configured to measure sodium, pH, potassium, glucose, lactate, or combinations thereof.

As also described herein, in some embodiments, the monitoring patch may also include a temperature sensor. In some embodiments, the monitoring patch may also include an electrocardiogram (ECG) surface electrode.

As further described herein, in some embodiments, the monitoring patch may include a lactate sensor. In some embodiments, the lactate sensor may include a subcutaneous lactate sensor. In some embodiments, the substrate may comprise a first side and a second side opposite the first side, where the lactate sensor is positioned on the first side of the substrate.

As further described herein, in some embodiments, the monitoring patch may also include a power source or a connection for a power source, which may be disposed on the second surface. As described herein, in some embodiments, the monitoring patch may include one or more impedance cardiology electrodes disposed in and/or on the substrate. In some embodiments, the one or more impedance cardiology electrodes may be disposed on the second surface. In some embodiments, each impedance cardiography electrode may have a portion on the first surface and a portion on the second surface of the substrate.

As also described herein, in some embodiments, the monitoring patch may include one or more sensors arranged to measure oxygen saturation. In some embodiments, the one or more sensors arranged to measure oxygen saturation may include NIR emitter/detectors, IR emitter/detectors, or both.

As also described herein, in some embodiments, the substrate may be configured to be bilaterally symmetrical, having a left portion and a right portion. In some embodiments, each impedance cardiography electrode may have a portion on a first surface of the substrate and a portion on a second surface of the substrate, the second surface being opposite the first surface. As described herein, in some embodiments, the substrate may be configured to be bilaterally symmetrical, having a left portion and a right portion, where a first impedance cardiography electrode is within the left portion and a second impedance electrode is within the right portion.

As still further described herein, in some embodiments, the substrate may be comprised of two or more layers.

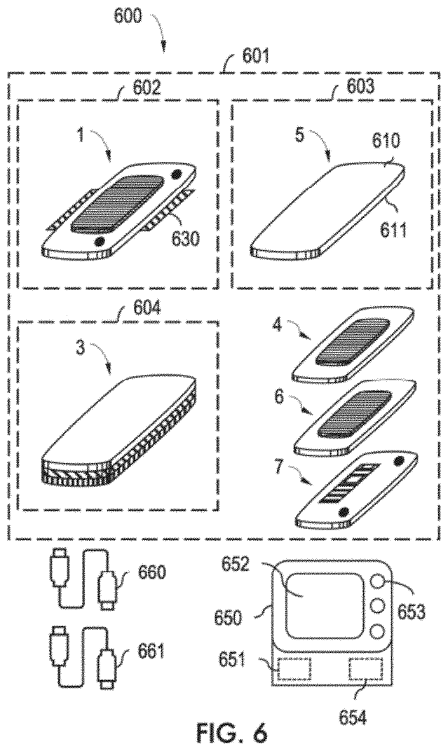

As seen in FIG. 6, some embodiments of the present disclosure include a monitoring patch kit 600. As shown in this view, the monitoring patch kit 600 may comprise or consist of a plurality of patches 1, 5, 3, 4. In some embodiments, a container 601 may be used to contain each of the patches in the kit. In some embodiments, the container 601 may include a sterile package. In some embodiments, some or all patches in the kit may be contained within its own separate compartment 602, 603, 604. In some embodiments, each of the separate compartments 602, 603, 604 may include a sterile package.

In some embodiments, the plurality of patches may include a first patch 1 comprising a monitoring patch according to any of the embodiments described herein, and a second patch 5. In some embodiments, the second patch 5 includes a substrate 610 having an adhesive surface 611 and a plurality of different sensors disposed in and/or on the substrate of the second patch, where at least one of the plurality of sensors disposed in and/or on the substrate of the first patch 1 is different from any of the plurality of different sensors disposed in and/or on the substrate of the second patch 5. For example, as seen in FIG. 6, the first patch 1 is shown as having a strain gauge 630, which is not present on the second patch 5. Other arrangements are obviously envisioned; for example, first patch 1 may comprise the sensors seen in FIG. 5E, while second patch 5 may comprise the sensors seen in FIG. 5A.

In some embodiments, the kit 600 may include a third patch 3 and a fourth patch 4, the third patch and the fourth patch each, independently, comprising a monitoring patch according to any of the embodiments described herein. In some embodiments, each of the third and fourth patches may comprise a substrate having an adhesive surface and a plurality of different sensors disposed in and/or on the substrate of each of the third and fourth patch, and at least one of the plurality of sensors disposed in and/or on the substrate of the first patch being different from any of the plurality of different sensors disposed in and/or on the substrate of the third patch and from any of the plurality of different sensors disposed in and/or on the substrate of the fourth patch. As seen in FIG. 6, first patch 1 is shown as having a strain gauge 630, which is not present on either the third patch 3 or fourth patch 4.

In some embodiments, the configuration of the first, second, third, and fourth patch are each different. In some embodiments, at least two of the second, third, or fourth patch are identical. For example, a kit may comprise a first patch having a first configuration, and a second, third, and fourth patch each having a second configuration.

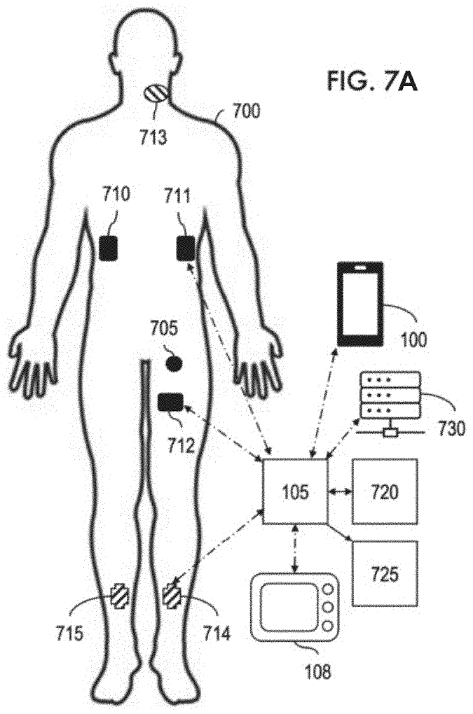

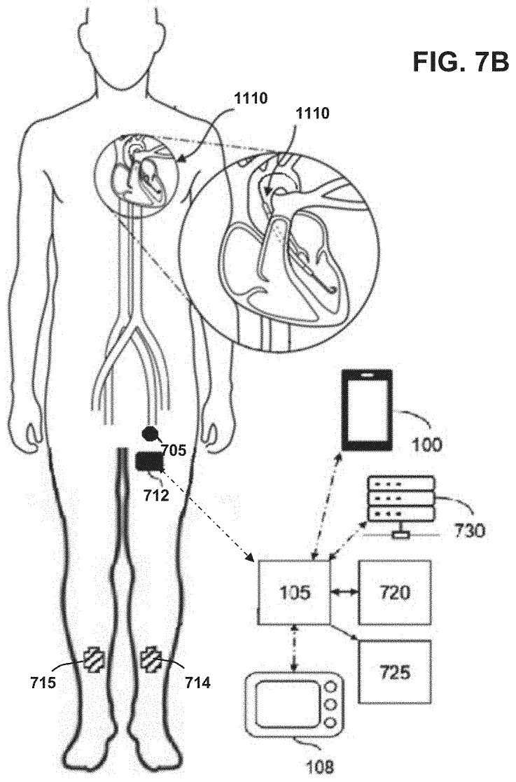

As shown in FIG. 7A, in some embodiments, monitoring patches 710, 711, 712, 713, 714, and 715 may be configured to be placed on different portions of the body of a subject 700. In some embodiments, the first patch (e.g., patch 710, 711, or 712) is configured to be placed on a first portion of a subject's body and the second patch (e.g., patch 713, 714, or 715) is configured to be placed on a different portion of the subject's body. In some embodiments, the first portion of the subject's body is a torso, thigh, or a combination thereof (e.g., patches 710, 711, and 712 are shown as being present on the subject's torso and thigh). In some embodiments, the different portion is a forearm, wrist, neck, calf, ankle, or combination thereof (e.g., patches 713, 714, and 715 are shown as being present on the subject's neck and calves).

In some embodiments, the first or second patch (e.g., patch 710) is configured to be placed on a subject's torso, upper arm, thigh, neck, or combination thereof, and wherein the other of the first or second patch (e.g., patch 714) is configured to be placed on a subject's forearm, wrist, calf, ankle, or combination thereof.

In some embodiments, the monitoring patch kit comprises at least two patches configured to be placed on a torso (e.g., patches 710 and 711), and at least one patch configured to be placed on a thigh (e.g., patch 712).

In some embodiments, the monitoring patch kit comprises at least one patch (e.g., patch 714) configured to be placed on a left calf or ankle, and at least one patch (e.g., patch 715) configured to be placed on a right calf or ankle.

In some embodiments, the monitoring patch kit comprises at least one patch configured to be placed on a neck.

In some embodiments, a surgical insertion site 705 (see FIGS. 7A and 7B) may exist, through which one or more medical devices are inserted into the subject's body. For example, as shown in FIB. 7B, one or more patches may be used with a patient receiving mechanical circulatory support (1110). In such embodiments, the one or more patches (e.g., patch 712, 714, 715) may be configured to monitor the patient, such as to measure a patient's temperature, heart rate and/or oxygen's saturation of surrounding tissue. As described herein, the sensed values can be displayed (e.g., on a patient console) and/or sent to a clinician. For example, the one or more sensors may provide information about the patient and regarding the operation of the pump providing mechanical circulatory support. As also described herein, the sensed values can be compared to acceptable predetermined ranges, and alerts may be provided to the clinician. In some embodiments, one or more processors may be operably communicating (e.g., receiving information from, sending information to, or a combination thereof) with both the monitoring patches and a mechanical circulatory support device.

As will be appreciated in view of the above, in some embodiments, the patches 710, 711, 712, 713, 714, and 715 of the kit 700 may each include the same number and type of sensors. In other embodiments, the patches may include more than one type of patch.

In some embodiments, the various monitoring patches may allow for simplified monitoring of hemodynamics and vitals, including monitoring cardiac output (e.g., via impedance cardiography), pulse pressure (e.g., via NIR sensors), oximetry (e.g., via NIR sensors), vascular resistance (e.g., via NIR sensors, and changes in pulse propagation across the body), heart rate (e.g., via ECG leads), and ECG (e.g., via ECG leads).

In addition, more advanced monitoring may occur. For example, local infection detection (e.g., via temperature sensors, such as those placed near insertion site), limb ischemia detection (e.g., via NIR sensors placed distal to insertion site), end organ perfusion readout (e.g., via lactate sensors), arrhythmia detection (e.g., via ECG leads), or measuring ascites (e.g., via strain gauges), or general movement of the patient (e.g., via an accelerometer).

In some embodiments, a patch 713 containing NIR sensors positioned on the neck of a subject may be used to add a measurement representative of brain oxygenation.

In some embodiments, duplicate or triplicate lactate and analyte measurements from patches 710, 711, and/or 712 may be used to provide real-time data on measures of end organ perfusion.

In some embodiments, multiple impedance cardiography measurements from patches 710, 711, 712, 714, and 715 may allow for cardiac output measurements.

In some embodiments, NIR measurements may be used, allowing for pulse oximetry, pulse pressure, and measurement of vascular resistance.

In some embodiments, patient movement may be detected and monitored. For example, by using the accelerometers, it may be possible to alert medical staff if a patient has moved too much or is not at an acceptable angle in a bed.

Referring again to FIG. 6, in some embodiments, the monitoring patch kit 600 may also include one or more additional patches 6, 7. In some embodiments, the one or more additional patches may comprise or consist of a first additional patch 6 having a first set of different sensors disposed in and/or on a first substrate, and a second additional patch 7 having a second set of different sensors disposed in and/or on a second substrate. The first set of different sensors may be different than the second set of different sensors, the first substrate may be different from the second substrate, and/or a shape of the first substrate may be different than a shape of the second substrate. FIG. 6 shows the first additional patch 6 having a different set of sensors than the second additional patch 7, but it will be recognized that other variations are easily produced.

The monitoring patch kit 600 may also include a controller 650. The controller 650 may include one or more processors 651. The one or more processors 651 may be operable connected to a display 652 and/or one or more buttons or controls 653. The controller 650 may be configured to receive data from one or more sensors on each of the plurality of patches. In some embodiments, the controller 650 may be operably connected to a wireless transceiver 654. In some embodiments, the one or more processors 651 may be operably connected to the wireless transceiver 654. In some embodiments, the patches may transmit raw sensor data to the controller. In some embodiments, the sensors may provide data to a processor on each respective patch, and the processor may calculate a value (such as heart rate) based on the raw sensor data, and the calculated value may then be transmitted to the controller.

The monitoring patch kit 600 may also include a plurality of leads 660, 661. Each lead 660, 661 may be configured to operably connect at least one sensor on one of the plurality of patches to the controller. In this manner, even if the monitoring patches cannot communicate with the controller wirelessly, sensor information can be passed along to the controller.

As will be appreciated, communication of sensor information may occur in a variety of ways. For example, in some embodiments, some or all of the patches are connected by leads to the controller. In other embodiments, some or all of the patches communicate wirelessly directly with the controller. In some embodiments, a first patch may communicate wirelessly directly with the controller, while the other patches communicate with the controller via the one or more leads. In other embodiments, the first patch may communicate wirelessly directly with the controller while the other patches communicate with the first patch. In such embodiments, the other patches may communicate with the controller via the first patch. For example, as shown in FIG. 2, first patch 1 may communicate with a controller 105 (which may include a processor 106 and a wireless transceiver 107). First patch 1 also may communicate with second patch 2. As will be appreciated, although the second patch 2 is shown as only communicating with first patch 1, the second patch 2 could also be configured to communicate with any of the components shown as communicating with first patch 1 (or to another patch).

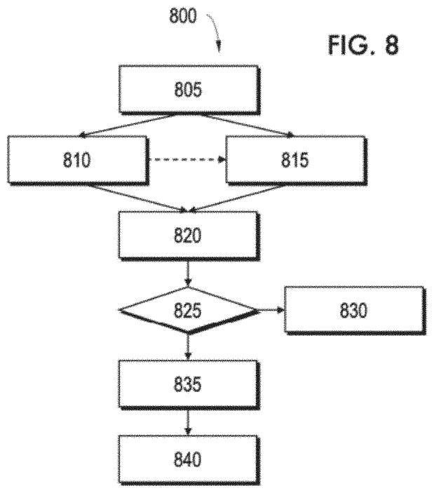

According to another embodiment, a method of using the monitoring patches is disclosed. This can be understood with reference to FIGS. 7A and 8. In some embodiments, the method 800 may include receiving 805 data from one or more sensors on one or more monitoring patches according to any of the embodiments described herein, the one or more monitoring patches being positioned on a subject. FIG. 7A illustrates sensors on at least patches 711, 712, and 714 communicating with a controller 105, and controller 105 may be receiving data from those sensors. In some embodiments, additional steps may occur in the method. As will be understood, these additional steps may be performed in any order.

In some embodiments, the method 800 may also include storing 810 the received data on one or more non-transitory computer readable media (e.g., non-transitory computer readable media 720, on a remote server, such as a database on a cloud-based server, etc.), displaying data 815 (e.g., the received data from the sensors and/or values derived from the received data) on a display (e.g., a secondary display 725, and/or a display screen on patient console 108), or a combination thereof. This may occur at any time after the data is received. In some embodiments, the displayed value may be an oxygenation level, a lactate level, a glucose level, a potassium level, a sodium level, a pulse rate, a pH, a temperature, and/or a difference between two received data points.

In some embodiments, data may be sent to the display after being received by the non-transitory computer readable media.

In some embodiments, the method 800 may also include transmitting 820 the data to a remote server (e.g., remote server and/or databases 730), a mobile device (e.g., smart phone 100), a patient console (e.g., patient console 108), or a combination thereof. This may occur at any time after the data is received.

In some embodiments, the method 800 may also include processing the data from at least one of the one or more sensors. For example, the method may include determining 825 whether the data received, or a value calculated from the data received, is outside a predetermined value or range.

In some embodiments, the step of determining may include comparing a first value from a first sensor against a predetermined value or range. For example, in some embodiments, data from the temperature sensors may be expected to provide temperatures within a predetermined range (e.g., a normal temperature range for the monitored patient). As will be appreciated, in such an example, if the temperature is determined to be outside the predetermined range, it may be indicative of infection.

In some embodiments, if the data is showing a trend, the slope or other variable representative of the trend may be used to evaluate the patient (e.g., if the slope or other variable representative of the trend is outside an acceptable predetermined range). As an example, in some embodiments, the skin temperature may have a predetermined range of between, e.g., 92° F. and 99° F. In some instances, if the measured temperature increases from 93° F. to 95° F. an alert may not be triggered. However, in other instances, if that 2-degree rise in temperature occurs over a prescribed period of time, such as 10 minutes, that rate of change (e.g., 1 degree every 5 minutes) may be outside a predetermined range, and an alert may be generated.

In other embodiments, the step of determining may include comparing a first value of the first sensor with a second value from a second sensor. For example, the method may include determining a first difference between a first value from a first sensor on a first patch and a second value from a second sensor on a second patch, at a first point in time. In some embodiments, the first value is a first pulse value, and a second value is a second pulse value.

In some embodiments, the step of determining may include determining a second difference between the first value from the first sensor measured at a second point in time and the second value from the second sensor measured at the second point in time.

In other embodiments, the step of determining may include comparing whether a difference between sensor values is within acceptable limits. For example, the method may include determining whether the first difference is outside a second predetermined range. For example, by comparing pulse oximetry data from patches 714 and 715 allow for the detection of limb ischemia when medical devices have been inserted through the insertion site 705. In some embodiments, the limb that does not contain the insertion site may be used as a control.

In other embodiments, the step of determining may include comparing tissue oxygenation data from patches 714 and 715 to allow for detection of limb ischemia when a medical device has been inserted through the insertion site. In some embodiments, the step of determining includes comparing each of the sensed values against a predetermined value or baseline (e.g., 70%). In some embodiments, a possible ischemic event may be determined if the tissue oxygenation is less than 60% for a predetermined period, such as for greater than 3 minutes. In some embodiments, a possible ischemic event may be determined if the sensed tissue oxygenation drops by more than 5% over a predetermined period, such as 30 minutes.

In some embodiments, the limb that does not contain the insertion site could be used as a control. For example, in one embodiment, if the difference in tissue oxygenation between the two limbs is greater than 20%, the step of determining may determine a possible ischemic event in the limb.

If the values (e.g., sensor data, calculated values based on the sensor data, differences, etc.) are outside a predetermined value or range, the method may include generating an alert 830, such as a visual signal or audio alert (e.g., a beep, tone, etc.). In some embodiments, the generation of alerts may be the only steps in the method 800 that cannot be performed in any order—they must follow a determination step (such as the step of determining 825) that at least some value is outside a predetermined range.

In some embodiments, the alert is intended to alert a subject and/or medical professional. In such embodiments, alerting the subject and/or medical professional may include generating an audible indicium, a visual indicium, a message (such as a text message, an email message, etc.), or a combination thereof. In some embodiments, the visual indicium includes a visual change to a display screen (such as a secondary display 725 and/or a display screen on patient console 108).

In some embodiments, the method 800 may also include determining and suggesting 835 a treatment plan based on the received data, the first difference, a change from the first difference to the second difference, or a combination thereof. For example, the controller 105 may receive the data, and based on a trained machine learning algorithm and treatments stored on a database on non-transitory computer readable media 720, a treatment plan can be automatically determined and suggested (e.g., displayed or otherwise conveyed to one or more people).

The subject may then be treated 840 based on the received data, or values derived from the received data, such as the first difference, a change from the first difference to the second difference, the suggested treatment, or a combination thereof.

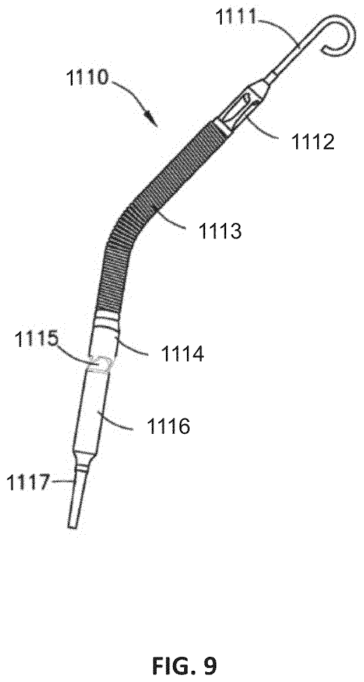

An example of a mechanical circulatory support device (also referred to as a “heart pump” or simply a “pump”) is shown in FIG. 9, and may include a percutaneous, catheter-based device that provides hemodynamic support to the heart of a patient. As shown in this view, a heart pump 1110 may include a pigtail 1111, an inlet area 1112, a cannula 1113, a pressure sensor 1114, an outlet area 1115, a motor housing 1116, and/or a catheter tube 1117. Pigtail 1111 may assist with stabilizing heart pump 1110 in the heart of a patient. It should be appreciated that some embodiments of heart pump 1110 may not include pigtail 1111 and heart pump 1110 may be stabilized in other ways or not at all. During operation, blood may be drawn into one or more openings of inlet area 1112, channeled through cannula 1113, and expelled through one or more openings of outlet area 1115 by a motor (not shown) disposed in motor housing 1116. In some implementations, pressure sensor 114 may include a flexible membrane that is integrated into cannula 1113. One side of pressure sensor 114 may be exposed to the blood pressure on the outside of cannula 1113, and the other side may be exposed to the pressure of the blood inside of cannula 1113. In some such implementations, pressure sensor 1114 may generate an electrical signal proportional to the difference between the pressure outside cannula 1113 and the pressure inside cannula 1113. In some implementations, pressure sensor 114 may include an optical pressure sensor. Catheter tube 1117 may provide one or more fluidic and/or electrical connections between heart pump 1110 and one or more other devices of a ventricular support system.

Those skilled in the art will recognize or be able to ascertain using no more than routine experimentation many equivalents to the specific embodiments of the invention described herein. Such equivalents are intended to be encompassed by the following claims.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A monitoring patch comprising:
a substrate having an adhesive surface;
a plurality of sensors disposed in and/or on the substrate, wherein the plurality of sensors includes:
one or more sensors arranged to measure oxygen saturation;
a lactate sensor; and
one or more impedance cardiography electrodes.

2. The monitoring patch according to claim 1, further comprising a sweat biosensor array.

3. The monitoring patch according to claim 2, wherein the sweat biosensor array is configured to detect Na⁺, K⁺, or a combination thereof.

4. The monitoring patch according to claim 2, wherein the sweat biosensor array is configured to detect at least pH, glucose, lactate, an alkali metal ion, an alkaline earth metal ion, or a combination thereof.

5. The monitoring patch according to claim 2, wherein the sweat biosensor array is configured to measure sodium, pH, potassium, glucose, lactate, or a combination thereof.

6. The monitoring patch according to claim 1, wherein the lactate sensor includes a subcutaneous lactate sensor.

7. The monitoring patch according to claim 6, wherein substrate comprises a first side and a second side opposite the first side, and wherein the lactate sensor is positioned on the first side of the substrate.

8. The monitoring patch according to claim 7, further comprising a power source or a connection for a power source disposed on the second side.

9. The monitoring patch according to claim 7, wherein the one or more impedance cardiography electrodes are disposed on the second side.

10. The monitoring patch according to claim 1, further comprising a temperature sensor.

11. The monitoring patch according to claim 1, further comprising an electrocardiogram (ECG) surface electrode.

12. The monitoring patch according to claim 1, further comprising a strain gauge.

13. The monitoring patch according to claim 1, further comprising an accelerometer.

14. The monitoring patch according to claim 1, wherein the one or more sensors arranged to measure oxygen saturation include NIR emitter/detectors, IR emitter/detectors, or both.

15. The monitoring patch according to claim 1, further comprising circuitry on a second side of the substrate, the second side being opposite a first side, the plurality of sensors comprising at least one sensor disposed in and/or on the first side, the circuitry being operably connected to the at least one sensor disposed in and/or on the first side and comprising a connector for connecting to a power source.

16. The monitoring patch according to claim 15, wherein the power source is operably coupled to the substrate.

17. The monitoring patch according to claim 16, wherein the power source is remotely located from the patch.

18. The monitoring patch according to claim 15, further comprising a detachable wireless transmitter or transceiver configured to operably connect to the circuitry.

19. The monitoring patch according to claim 18, wherein the detachable wireless transmitter or transceiver is a detachable Bluetooth transmitter or transceiver.

20. The monitoring patch according to claim 1, wherein the monitoring patch is configured to connect to a mobile phone, a tablet, or a desktop computer.

21. The monitoring patch according to claim 1, wherein the monitoring patch is configured to operably communicate with a remote server.

22. The monitoring patch according to claim 1, wherein the substrate is configured to be bilaterally symmetrical, having a left portion and a right portion.

23. The monitoring patch according to claim 1, where each impedance cardiography electrode comprises a portion on a first surface and a portion on a second surface of the substrate, the second surface being opposite the first surface.

24. The monitoring patch according to claim 23, wherein the substrate is configured to be bilaterally symmetrical, having a first portion and a second portion opposite the first part, and wherein a first impedance cardiography electrode is positioned within the first portion and a second impedance electrode is positioned within the second portion.

25. The monitoring patch according to claim 1, wherein the substrate is comprised of two or more layers.

26. A monitoring patch comprising:
a substrate having an adhesive surface;
a plurality of sensors disposed in and/or on the substrate, wherein the plurality of sensors includes:
an accelerometer; and
a strain gauge,
wherein the monitoring patch is free of a sensor arranged to measure oxygen saturation, a lactate sensor, an impedance cardiography electrode, or a combination thereof

27-51. (canceled)

52. A system for combination use, comprising:
a monitoring patch of claim 1 operably coupled to a patient; and
a mechanical circulatory support device operably coupled to the patient.

53. (canceled)

54. A monitoring patch kit comprising: a plurality of patches comprising:
a first patch comprising a monitoring patch according to claim 1; and
a second patch.

55-69. (canceled)

70. A method, comprising:
receiving data from one or more sensors on one or more monitoring patches according to claim 1, the one or more monitoring patches being positioned on a subject.

71-88. (canceled)
